(19) United States
(12) Patent Application Publication
(10) Pub. No.: US 2026/0018269 A1
(43) Pub. Date: Jan. 15, 2026

(54) Speech-based recognition of emotions reported and detected along with concordances and discrepancies

(71) Applicant: Colleen Elizabeth Crangle, Palo Alto, CA (US)

(72) Inventor: Colleen Elizabeth Crangle, Palo Alto, CA (US)

(21) Appl. No.: 18/769,282

(22) Filed: Jul. 10, 2024

(51) Int. Cl. G16H 15/00; G06F 40/279; G10L 15/26; G16H 10/60

(52) U.S. Cl. CPC G16H 15/00; G06F 40/279; G10L 15/26; G16H 10/60

(57) ABSTRACT

The present disclosure relates to the recognition of emotion in speech, both what is said and how it is said and the detection of a possible concordance or discrepancy between the two. A method is described for generating a time-stamped history of emotions a user reports by voice along with emotions detected automatically from those voice reports. A user utterance is analyzed using speech-to-text processing and a natural-language processing model to determine the emotion the user reports feeling. The user utterance is also analyzed using acoustic analysis to detect the emotion expressed in the user's voice report. A harmony report is generated from the time-stamped reports of concordance and discrepancy to measure the extent to which the user's perception of their emotions agrees with the emotions detected. The purpose of the invention is to provide insight into a user's emotions in real time and over time.

### FEDERALLY SPONSORED RESEARCH

This work was made with government support under Grant Number 1R41LM012177-0 awarded by the National Institutes of Health. The government has certain rights in the invention.

### INTERNATIONAL CLASSES

Primary Class
G10L 25/00
Secondary Classes
G10L 25/27; G10L 25/30; G10L 25/48; G10L 25/51; G10L 25/63; G06F 17/00

### FIELD OF THE DISCLOSURE

The disclosure herein generally relates to voice analysis techniques and systems, and, more particularly, to the recognition of human emotional states through speech along with the analysis thereof.

### BACKGROUND

Keeping a log of emotions over extended periods has long been a practice in therapeutic models such as cognitive-behavioral therapy. It is also a personal practice for individuals who want insight into their emotional functioning individually or within a relationship. The disclosure reported in this application provides a way to keep a log of emotions through the individual using their voice to report what they are feeling. What the individual learns from such a log, however, will depend on the extent to which their insight is accurate. A person's spoken report of “I'm fine today” may reveal acoustic markers of Tension or Sadness, for example. That is, what is said and the way it is said may reveal a discrepancy. Alternatively, the self-report of an emotion and the way that self-report is delivered by voice may be in concordance, with the individual accurately perceiving the emotion they are experiencing and expressing in their voice.

Insight into a person's own emotions plays a role in the understanding and management of mental health. Studies have examined the relationship between emotion-regulation strategies (such as reappraisal and rumination) and disorders such as anxiety, depression, and eating and substance-abuse disorders. Strategies for regulating emotion, however, depend on the individual's accurate understanding of their own emotions. One or more aspects of the disclosure described herein may improve the understanding and management of mental health by giving an individual a better understanding of the accuracy of their perception of their emotions.

The role of emotion in self-knowledge is not yet well understood, as laid out in Montes Sánchez and Salice's 2023 book “Emotional Self-Knowledge.” To date, self-knowledge inquiries have focused overwhelmingly on cognitive states. One or more aspects of the disclosure described herein may explain the roles that emotions play in promoting or obstructing our knowledge of ourselves and thereby explicate the role that self-knowledge plays in mental wellness.

Insight into a person's own emotions plays several roles in the wellness industry. Such insight may be aimed at improving mental wellness in meditation and fitness apps, in health and stress management programs for employees, and in assessing emotional compatibility within dating applications. One or more aspects of the disclosure described herein may provide new products and services through personalization related to emotions.

Automatic speech recognition has progressed to the point that voice interaction with devices is part of daily life. Speech-to-text techniques in speech recognition systems identify the words spoken by a human user based on various qualities of a received audio input. Computers, hand-held devices, smartphones, smart watches, telephone computer systems, and a wide variety of other devices can use microphone technology to enable speech recognition to interpret what a human user is saying.

Microphone technology in these devices also enables the capture of a digital audio sample of the speech. Automatic emotion detection entails an analysis of the acoustic data of the digital audio sample to extract features that are relevant to how emotions are expressed and then analyzing those features to determine the emotion being expressed in the speech sample.

Theoretical conceptualizations of emotions are of two main kinds. The first represents emotions as discrete categories such as Happiness and Sadness, with several emotion categories broadly considered basic, namely Happiness, Sadness, Surprise, Fear and Anger. An argument for basic emotions can be found in the Ekman's 1992 paper “An argument for basic emotions.” Emotions may also be represented dimensionally. Russell's 1980 paper “A Circumplex Model of Affect” lays out a psychological model that represents emotions along continuous quality dimensions, including valence and arousal. Valence can range from positive (pleasant) to negative (unpleasant) emotional states, while arousal can range from high (excited, activated) to low (calm, deactivated) emotional states.

Computer algorithms for automatically detecting emotion in the voice exist for both categorical representations of emotions and dimensional representations. A trained multi-label classification neural network can take digital audio input and output a list of emotion categories along with an estimate of the confidence with which the algorithm detects each of the emotion categories in the digital audio input. Examples are described in Lieskovska et al.'s 2021 paper “A Review on Speech Emotion Recognition Using Deep Learning and Attention Mechanism,” Alternatively, a trained multi-label classification neural network can take digital audio input and, for each element of a set of selected dimensions, such as valence or arousal, output a score representing the point at which the digital audio input lies on the relevant continuum. Yang & Hirschberg's 2018 article “Predicting Arousal and Valence from Waveforms and Spectrograms using Convolutional Neural Networks and Bidirectional Long Short-Term Memory Networks” provides examples.

Trained multi-label classification neural networks for emotion recognition are a more recent development from other kinds of trained multi-label machine learning methods for the recognition of emotion in speech. An example of an earlier method can be found in Crangle et al.'s 2019 paper “Machine learning for the recognition of emotion in the speech of couples in psychotherapy using the Stanford Suppes Brain Lab Psychotherapy Dataset.” A review that discusses both older and newer methods of emotion recognition in speech can be found in de Lope and Grana's 2023 article “An ongoing review of speech emotion recognition.”

Generative Artificial Intelligence (AI) Large Language Models (LLMs) have recently been made available to developers. Access to these LLMs is through application programming interfaces (APIs). A widely available series of LLMs is given by GPT, short for Generative Pre-trained Transformer. These language models, developed by OpenAI and evolved from GPT-1 to GPT-4, are trained on vast text data and can be further refined for specific language tasks thought API calls. LLMs excel in generating coherent text by interpreting a natural-language prompt given to them and predicting appropriate words for their response to the prompt. ChatGPT, a conversational AI based on the GPT models, provides access to OpenAI's conversational AI models through an API. Brown et al.'s 2020 paper “Language Models are Few-Shot Learners” describes the development of GPT-3, the architecture behind ChatGPT.

Attempts have been made to probe differences between self-reported and expressed or perceived emotions. In Zhang et al.'s 2016 paper “Automatic Recognition of Self-Reported and Perceived Emotion: Does Joint Modeling Help?”, the mismatch between self-reported and perceived emotions was investigated. However, the perception being investigated was that by others, not by the person expressing or reporting the emotion. The aim was to provide better labeling of emotion database samples by combining self- and other-perceived emotion reports. In the disclosure described herein, the individual's self-report is what they perceive their own emotion to be; said perception is contrasted with the emotion automatically detected in the audio of the self-report. Furthermore, distinctions between the two are pursued in several aspects of the disclosure herein to support a range of applications in wellness and mental health, not merely to provide a way to label database samples. In Mettler et al.'s 2021 paper “Perceived vs. Actual Emotion Reactivity and Regulation in Individuals with and Without a History of NSSI”, the accuracy of self-reported emotion regulation strategies such as reactivity was explored experimentally. However, no attention was paid to the direct measurement, analysis and evaluation of self-reported emotions in comparison to automatically detected emotions in the voice. The simultaneous identification of a self-reported emotion and an acoustically detected emotion and the comparison thereof confers an advantage on the present disclosure.

The novel combination of self-reported emotions with emotions detected in those self-reports overcomes the disadvantage of using self-reports alone in multiple aspects of the present disclosure. Self-reports have the disadvantage of possibly not accurately capturing the emotion being experienced and expressed in the voice.

### BRIEF DESCRIPTION OF THE DRAWINGS

Other objects and advantages of the present disclosure will become apparent to those skilled in the art upon reading the following detailed description of an exemplary and other embodiments, in conjunction with the accompanying drawings, wherein like reference numerals have been used to designate like elements, elements depicted in multiple figures are designated with consistent reference numbers, elements are numbered according to the figure in which they first appear, and wherein:

FIG. 1 depicts a context of use in which a user reports their emotion to a mobile device and a digital output report appears on the screen of the device showing what the user said, what emotion the user reported feeling, what emotion the system detected in the user's voice, and whether there was a concordance or a discrepancy between the Reported and Detected Emotions. An ordinary-language or natural-language interpretation of the Concordance-Discrepancy Report is also displayed.

FIG. 2 depicts a context of use in which a user reports what they are feeling at a given time and, after a period of time has elapsed and the user has reported multiple instances of what they are feeling at different times, the output is displayed on the mobile device for the Reported and Detected Emotions and Concordance-Discrepancy Reports.

FIG. 3 depicts a context of use in which a mobile device displays a prompt for the user to report what they are feeling.

FIG. 4. is an overview flowchart; it depicts a flow diagram for recognizing a Reported Emotion, a Detected Emotion, and a concordance or a discrepancy between the Reported and Detected Emotions, accompanied by an ordinary-language interpretation of the concordance or discrepancy.

FIG. 5. depicts a flow diagram for recognizing a Reported Emotion.

FIG. 6 depicts a flow diagram for a natural-language processing model for recognizing a Reported Emotion.

FIG. 7 depicts a flow diagram for generating a Detected Emotion.

FIG. 8A depicts a 2-step flow diagram for emotion detection model A.

FIG. 8A (i) depicts a flow diagram for step 1 of FIG. 8A, namely identifying an Emotion Category from an acoustic feature vector

FIG. 8A (ii) depicts a flow diagram for step 2 of FIG. 8A, namely identifying Dimensional Emotion Qualities corresponding to an Emotion Category.

FIG. 8B. depicts a flow diagram for emotion detection model B.

FIG. 9 depicts a flow diagram for a concordance-discrepancy model.

FIG. 10 depicts a flow diagram for computing a harmony metric from a history of digital output reports.

FIG. 11 depicts a system chart showing a mobile device, a server, a connection between the mobile device and the server, the components of the mobile device and the server, and the operation of the components on the mobile device or server.

### DETAILED DESCRIPTION

The following terms are defined in the exemplary embodiment described herein. These technical terms are used in the sections that follow as more precise uses of terms used generally in the foregoing Background section.

The term ‘Reported Emotion’ as used herein refers to the emotion the user reports by voice as derived by the method from the user's spoken report, the transcript resulting from the transcription process applied to that spoken report, and the natural-language model processing of the transcript.

The term ‘Detected Emotion’ as used herein refers to the emotion the method detects in the digital audio sample derived from the user's spoken report of their emotion.

The term ‘Concordance-Discrepancy Report’ as used herein refers to the sequential combination of a timestamp indicating when the individual reported their emotion by voice; the transcript of what they said; the category name of the Reported Emotion; the category name of the Detected Emotion; and a mark of an asterisk (*) indicating a discrepancy between the Reported Emotion and the Detected Emotion or a tick (√) indicating a concordance between the Reported Emotion and the Detected Emotion.

The term ‘Emotion Category’ as used herein refers to those emotions broadly considered basic, namely Happiness, Sadness, Fear and Anger, and the emotion Fine, defined as lying between Happiness and Neutral but with low energy, and the emotion Tense, defined as closer to Sadness, Fear and Anger than to Happiness and with low energy, as well as Neutral, which is defined as the absence of the aforementioned emotions.

The term ‘Dimensional Emotion Qualities’ as used herein refers to the dimensions arising from the psychological model that represents emotions along continuous dimensions, most typically but not restricted to, the dimensions of valence and arousal, where valence ranges from positive (pleasant) to negative (unpleasant) emotional states and arousal ranges from high (excited, activated) to low (calm, deactivated) emotional states.

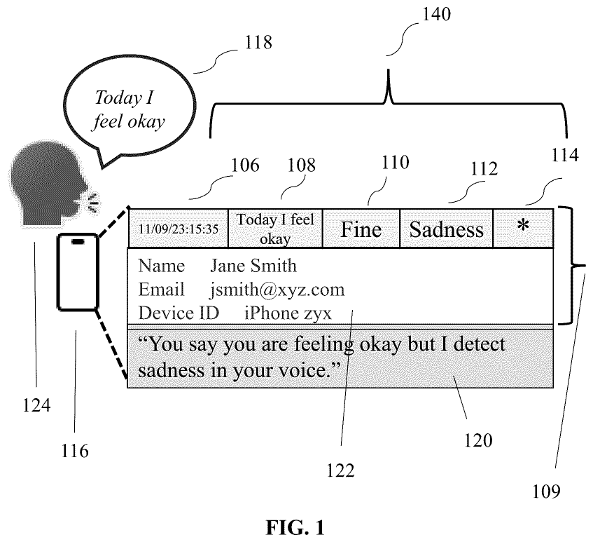

FIG. 1 depicts a context for an exemplary embodiment in which a user 124 reports what they are feeling (that is, their emotion) using the words “I feel” or “I am” or a syntactic or semantic equivalent by speaking into a mobile device 116 capable of sending and receiving voice calls and text messages. Syntactic equivalents of “I feel fine” are phrases such as “I am feeling fine” and semantic equivalents are phrases such as “I've never been so fine.” FIG. 1 further depicts the response generated by the methods disclosed herein: the transcript 108 which is the result of a text-to-speech system that uses automatic speech recognition to determine the words the individual has spoken 118; a timestamp 106 indicating when the individual reported their emotion; a Reported Emotion's category name 110; a Detected Emotion's category name 112; a concordance-discrepancy mark of an asterisk (*) 114 indicating a discrepancy between the Reported Emotion and the Detected Emotion. The discrepancy results from the user reporting being okay and the method detecting Sadness in the voice. The concordance-discrepancy mark can be a tick (√) indicating a concordance between the Reported Emotion and the Detected Emotion if such a relation holds. Entities 106, 108, 110, 112, and 114 together comprise a Concordance-Discrepancy Report 140. Entity 122—comprising the user's name, the user's email address, and a unique identifier of the user's device—and the Concordance-Discrepancy Report 140 collectively make up a digital output report 109. An Application Programming Interface (API) to a Generative Artificial Intelligence (AI) Large Language Model (LLM) may display an ordinary language interpretation of “You say you are feeling Fine but I detect Sadness in your voice” 120 of the Concordance-Discrepancy Report 140 for the individual 124 on the mobile device 116.

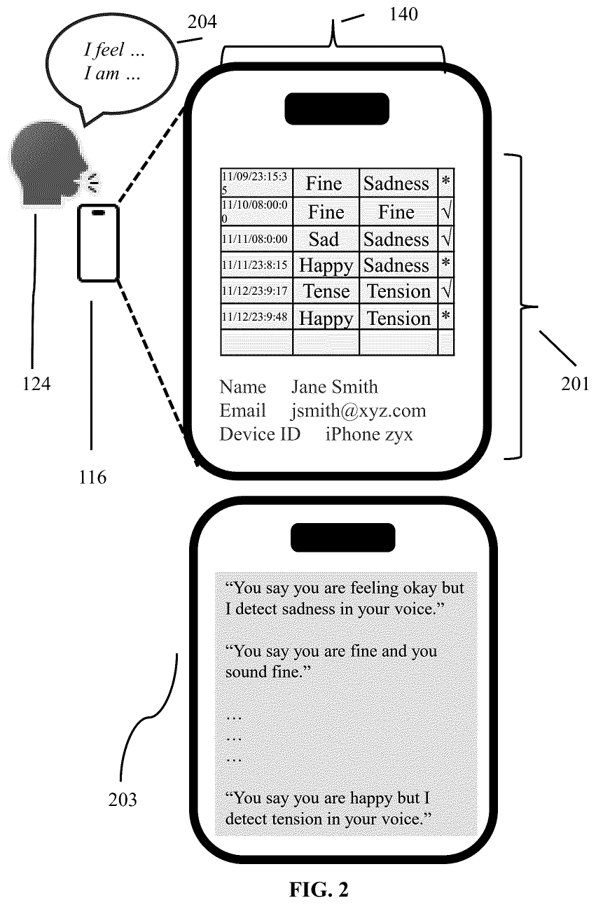

FIG. 2 depicts a context for an exemplary embodiment in which a user 124 reports what they are feeling in multiple instances over a period of time 204. The plurality of digital output reports arranged sequentially can be a history of digital output reports 201. The history of digital output reports 201 is displayed on the mobile device 116. On a follow-up screen, a plurality of Generative AI LLM interpretations 203 of the plurality of Concordance-Discrepancy Reports 140 in the history of digital output reports 201 is displayed.

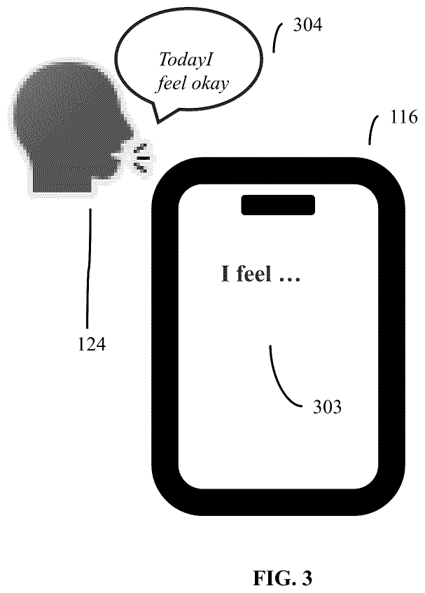

FIG. 3 depicts a context for an exemplary embodiment in which a mobile device 116 displays the carrier phrase “I feel . . . ” 303 as a prompt for a user 124 to report what they are feeling in an utterance 304 starting with “I feel” or “I am” or the syntactic or semantic equivalent. In other embodiments, the prompt may be an incoming phone call or incoming text message asking the individual what they are feeling or an outgoing call initiated by the individual to report their emotion.

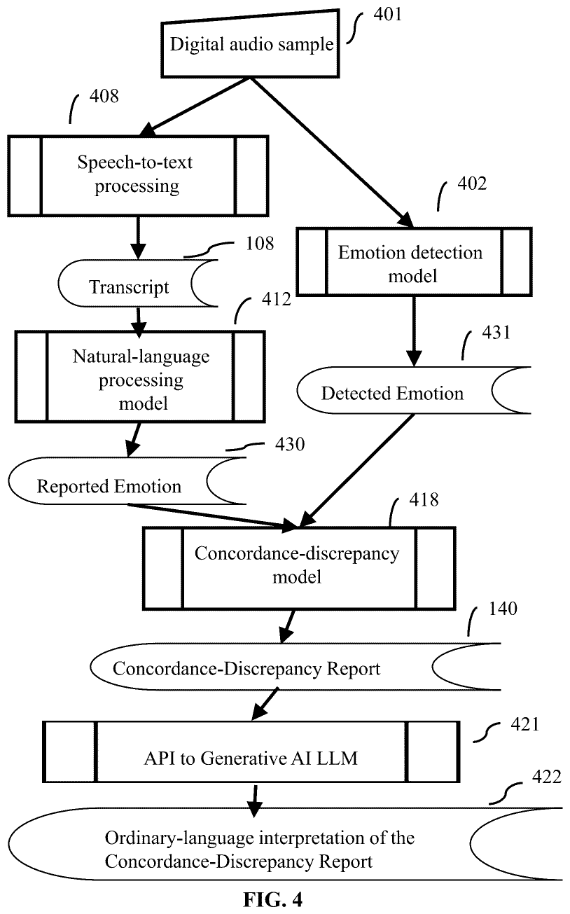

FIG. 4 is an overview flow diagram for an exemplary embodiment; it depicts a flow diagram for a computer-implemented method to generate a Reported Emotion 430, a Detected Emotion 431, and a Concordance-Discrepancy Report 140 arising from a user verbally reporting the emotion they are feeling. The individual's verbal report produces a digital audio sample 401 which is input to a speech-to-text processing system 408. The output from the speech-to-text system is a transcript 108 that consists of the words the individual has spoken, which are displayed on the mobile device associated with the user. The transcript 108 is input to a natural-language processing model 412 that produces a Reported Emotion 430 that is displayed for the individual on the mobile device. The digital audio sample 401 is also input to an emotion detection model 402, which produces a Detected Emotion 431 that is displayed for the individual on the mobile device. The Reported Emotion 430 and Detected Emotion 431 are input to a concordance-discrepancy model 418, which produces a Concordance-Discrepancy Report 140. The Concordance-Discrepancy Report 140 is input to an API to a Generative AI LLM 421 that produces an ordinary-language interpretation 422 of the Concordance-Discrepancy Report 140. The method may include the step of storing the digital audio sample 401 along with the Reported Emotion 430, Detected Emotion 431, and Concordance-Discrepancy Report 140 for later analysis.

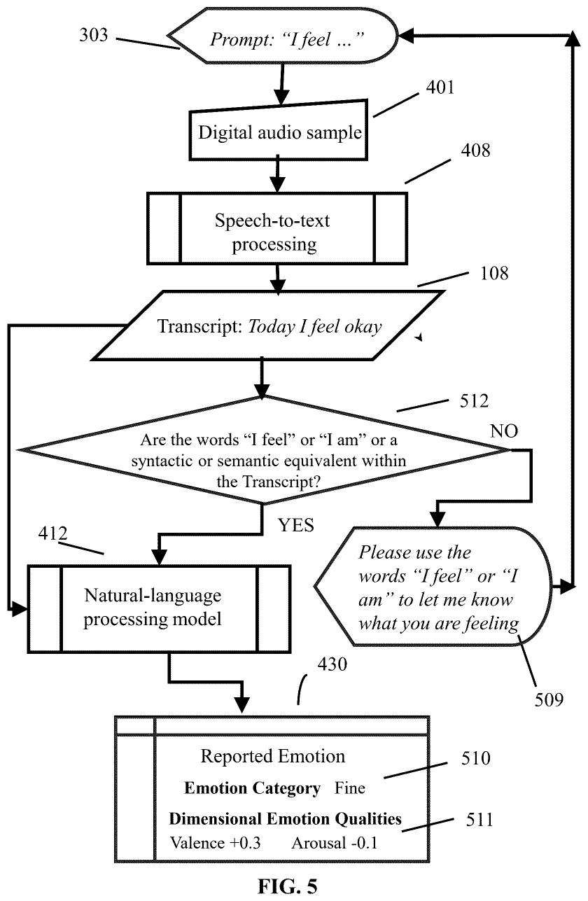

FIG. 5 depicts a flow diagram for an exemplary embodiment of a computer-implemented method to generate a Reported Emotion 430 from a digital audio sample 401 obtained from the user's response to a prompt of the carrier phrase “I feel . . . ” 303 displayed on the mobile device. In other embodiments, the prompt may use different words and it may be spoken, it may be an incoming phone call asking the individual what they are feeling, an outgoing call initiated by the user to report their emotion. In other embodiments, the prompt could be a text message asking the user to call a number to report what they are feeling.

In an exemplary embodiment, the digital audio sample 401 of the user saying “Today I feel okay” is input to a speech-to-text processing system 408, which produces the transcript 108 “Today I feel okay.” The computer-implemented method in an exemplary embodiment checks the transcript to make sure that the user is using the canonical way to verbally report their feelings by using a textual query to determine if the words “I feel” or “I am” are within the transcript 108. In the exemplary embodiment syntactic and semantic equivalents of “I feel” and “I am” are also checked for. In other embodiments a canonical form of reporting feelings may not be present; the transcript 108 of user's verbal report is simply passed directly to the natural-language processing model 412.

If the textual query 512 in the exemplary embodiment results in an answer of Yes, then the transcript 108 is input to a natural-language processing model 412. If the textual query results in an answer of No, then in an exemplary embodiment the following message 509 can be displayed on the digital device, under which the carrier phrase “I feel . . . ” 503 is displayed: ‘Please use the words “I feel” or “I am” to let me know what you are feeling’. The natural-language processing model 412 outputs a Reported Emotion 430, which comprises the Dimensional Emotion Qualities 511.

In the exemplary embodiment of FIG. 5, a predetermined set of Emotion Categories is used. The set comprises the emotions broadly considered basic, namely Happiness, Sadness, Fear and Anger, and the emotion Fine, defined as lying between Happiness and Neutral but with low energy, and the emotion Tense, defined as closer to Sadness, Fear and Anger than to Happiness and with low energy, as well as Neutral, which is defined as the absence of the aforementioned emotions. In other embodiments, other Emotion Categories can be used.

The Dimensional Emotion Qualities in the exemplary embodiment of FIG. 5 are valence and arousal. Valence ranges from positive (pleasant) to negative (unpleasant) emotional states, while arousal ranges from high (excited, activated) to low (calm, deactivated) emotional states. The values of the Dimensional Emotion Qualities can be converted to a range of [−1,+1], making them amenable to algorithmic computation. The Emotion Category Fine has an average valence value of +0.3 on a scale of −1 to +1 and an average arousal value of −0.1 on a scale of −1 to +1, indicating low arousal below but near the Neutral value of 0 and low valence above but near the Neutral value of 0. In other embodiments, other Dimensional Emotion Qualities can be used, such as dominance, which ranges from feelings of control and power (high dominance) to feelings of passivity and lack of control (low dominance) and can be used to differentiate emotions that have similar valence and arousal but differ in the sense of control or power, such as Anger (high dominance) versus Fear (low dominance).

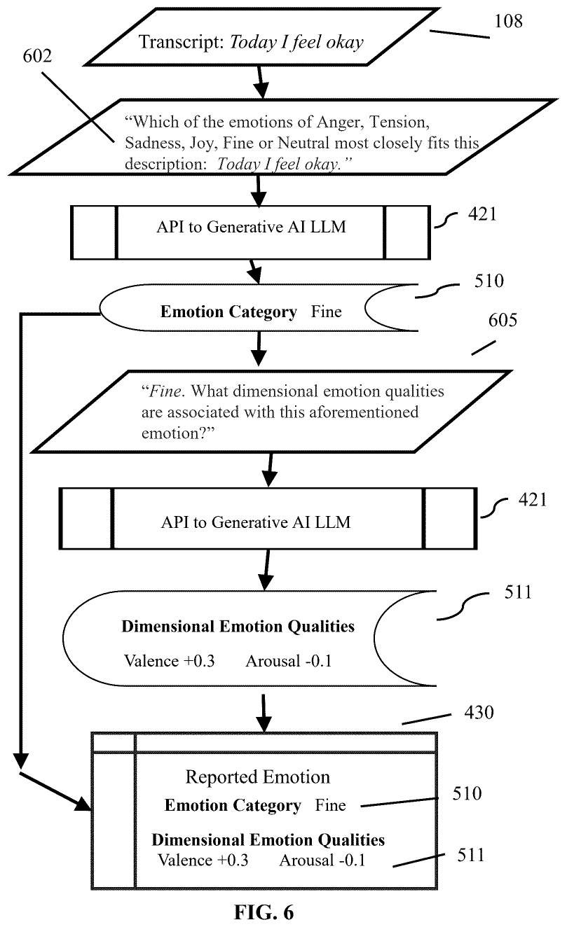

FIG. 6 depicts a flow diagram for a natural-language processing model in an exemplary embodiment that takes a transcript 108 comprising a written description of an emotion and produces a Reported Emotion 430, comprising an Emotion Category and Dimensional Emotion Qualities, representing the emotion described in the transcript 108. APIs to Generative AI LLMs can offer a way to transform an ordinary-language or natural-language report of an emotion by a user into emotion categories. The user's natural-language description of their emotion, as captured in the transcript 108, may be transformed into one or more of the predetermined set of Emotion Categories mentioned in reference to FIG. 5, namely the six Emotion Categories of Anger, Tension, Sadness, Joy, Fine and Neutral. The natural-language description is incorporated into a prompt to an API to a Generative AI LLM 421. For example, the natural-language description of the user's emotion, as captured in the transcript 108 “Today I feel okay,” can be incorporated into the prompt 602 to the API to the Generative AI LLM 421: “Which of the emotions of Anger, Tension, Sadness, Joy, Fine or Neutral most closely fits this description: Today I feel okay.?” The Emotion Category of Fine 610 is output from the API call to the Generative AI LLM 421 and displayed on the user's device. As another example, the prompt “Which of the emotions of Anger, Tension, Sadness, Joy, Fine or Neutral most closely fits this description? I am disappointed and unhappy.?” could produce the output of the Emotion Category of Sadness.

In the exemplary embodiment depicted in FIG. 6, an interface to ChatGPT 4o from OpenAi, accessible through the OpenAI Python API library (available at https://platform.openai.com/docs/api-reference/introduction?lang=python), permits the presentation of the prompt 602 as the value of the content parameter in a call to ChatGPT, which is an API to the Generative AI LLM specified as the value for the model parameter, namely ‘gpt-4-1106-preview’ in the Python code below.

```
from openai import OpenAI
chat_completion = client.chat.completions.create(
  messages=[
    {
      “role”: “user”,
      “content”: “Which of the emotions of Anger, Tension, Sadness, Joy, Fine or Neutral most
closely fits this description: Today I feel okay.? Report the result as a single word.”,
    }
  ],
  model=“gpt-4-1106-preview”,
)
```

In an exemplary embodiment, the output from the API to the Generative AI LLM 421 using ChatGPT with the prompt 602 for the transcript “I feel okay” is the single word “Fine.”

APIs to Generative AI LLMs can also offer a way to transform a named Emotion Category into dimensional emotion qualities such as valence and arousal. The output of the Category Fine 610 can form part of a prompt 605 to the API to the Generative AI LLM 421. The prompt may be: “Fine. What Dimensional Emotion Qualities are associated with this aforementioned emotion?”

An exemplary embodiment with ChatGPT 40 and the OpenAI Python API library uses the following Python code:

```
chat_completion = client.chat.completions.create(
  messages=[
    {
      ″role″: ″user″,
      ″content″: Fine. Convert the Dimensional Emotion Qualities of the aforementioned
emotion to a numeric scale, using the framework proposed by Russell's Circumplex Model of
Affect and using a scale from −1 to 1 for valence and arousal. Report the results as ‘Valence’
followed by a number and ‘Arousal’ followed by a number.”,
    }
  ],
  model=″gpt-4-1106-preview″,
)
```

The output from the API call to the Generative AI LLM 421 using the prompt 605 is the Dimensional Emotion Qualities 511 displayed as follows:

| | |
|---|---|
| Valence +0.3 | Arousal −0.1. |

The output of Dimensional Emotion Qualities 511 from the API call to the Generative AI LLM 421 makes up the Reported Emotion 430.

In an exemplary embodiment for FIG. 6, the six Emotion Categories of Anger, Tension, Sadness, Joy, Fine and Neutral have the Dimensional Emotion Qualities shown in the table below. These values can be produced by calls to the API to the Generative AI LLM 421.

| Emotion | Valence | | Arousal | |
|---|---|---|---|---|
| Category | Value | Reason for value | Value | Reason for value |
| Neutral | 0 | Neutrality is neither positive | 0 | a Neutral state is neither |
| | | nor negative | | highly aroused nor deeply |
| | | | | relaxed |
| Fine | +0.3 | Fine is Neutral to slightly | −0.1 | Fine suggests a calm, low- |
| | | positive | | activation state |
| Happy | +0.8 | Happiness is a strongly | +0.6 | Happiness is typically |
| | | positive emotion | | associated with moderate |
| | | | | to high arousal |
| Sad | −0.8 | Sadness is a strongly | −0.4 | Sadness is typically |
| | | negative emotion | | associated with low to |
| | | | | moderate arousal |
| Angry | −0.8 | Anger is a strongly negative | +0.7 | Anger is a typically |
| | | emotion | | associated with medium to |
| | | | | high arousal |
| Tense | −0.5 | Tension is generally an | +0.7 | Tension is typically |
| | | unpleasant emotion, but not | | associated with medium to |
| | | as negative as emotions like | | high arousal and alertness |
| | | Sadness or Anger | | |

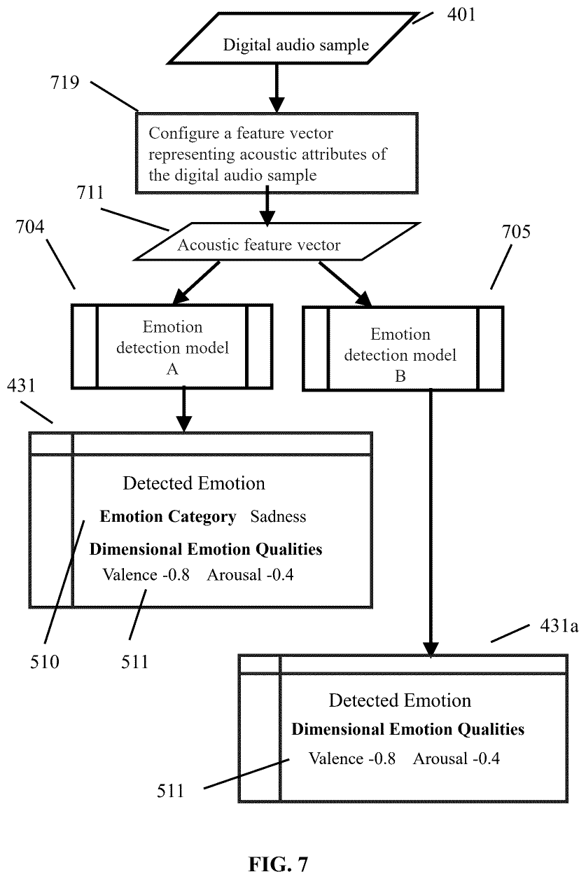

Although the above table lists values of valence and arousal for the six emotions of Neutral, Fine, Happy, Sad, Angry and Tense, other values may be assigned in other embodiments. FIG. 7 depicts a flow diagram of a computer-implemented method for generating a Detected Emotion 431 in an exemplary embodiment. The method takes a digital audio sample 401 and produces a Detected Emotion 431, comprising and Dimensional Emotion Qualities 511. From a digital audio sample 401 a set of acoustic attributes of the audio sample are derived and organized through a process 719 into an acoustic feature vector 711 that represents the digital audio sample 401. The acoustic feature vector 711 may be given as input to one of two alternative emotion detection models, A 704 or B 705. Although two emotion detection models are shown in the exemplary embodiment, other emotion detection models may be used.

Processing 719 the digital audio sample 401 in the exemplary embodiment of FIG. 7 to produce a set of relevant acoustic speech attributes can comprise first segmenting the data into voiced and unvoiced sounds, then extracting features such as pitch or fundamental frequency F0, and measures of voice quality such as formants F1, F2, F3 (frequency and bandwidth of energy peaks in the spectrum due to natural resonances of the vocal tract), speech rate, pauses, voice intensity, voice onset time, jitter (pitch perturbations), shimmer (loudness perturbations), voice breaks, and pitch jumps. These features are relevant to how emotions are expressed or perceived, as described in Myers-Schulz et al.'s 2013 article “Inherent emotional quality of human speech sounds.” For example, the center of gravity of the sound spectrum, the spectral centroid, is associated with an impression of how “bright” or pleasant a sound is. Formants, which appear as prominent peaks in the sound spectrum of a speech signal, are high-energy occurrences within the frequency spectrum that arise from resonances in the vocal tract and can be changed by moving lips, jaws, tongue, and soft palate. For the first two formants (F1 and F2), a lower F2 position, a smaller dispersion between F1 and F2 and an upward F1/F2 shift inherently sound lighter, faster and more pleasant or positive whereas a higher F2 position, greater dispersion between F1 and F2 and a downward F1/F2 shift inherently sound aversive or negative, for instance.

Formally, features in the exemplary embodiment may be compiled from the extended Geneva Minimalistic Acoustic Parameter Set (eGeMAPS), which is described in Eyber et al.'s 2016 article “The Geneva Minimalistic Acoustic Parameter Set (GeMAPS) for Voice Research and Affective Computing.” Said set consists at base of 18 low-level descriptors (LLDs) which, sorted by parameter groups, include frequency related parameters (pitch or logarithmic of the fundamental frequency F0 and jitter or pitch perturbations); energy/amplitude related parameters (loudness, shimmer or loudness perturbations, and harmonics-to-noise ratio); and spectral or balance parameters (alpha ratio, Hammarberg index, Formants 1, 2 and 3). LLDs may be smoothed over time with a symmetric moving average filter, and the arithmetic mean and coefficient of variation applied as functionals to all 18 LLDs, yielding 36 parameters. Further functionals may be applied along with some temporal features such as the number of loudness peaks per second and spectral (balance/shape/dynamics) parameters, including Mel-Frequency Cepstral Coefficients 1-4, resulting in a total of 88 parameters.

It is not always clear what features are effective for the task of recognizing emotions in speech. An alternative approach to using human-defined features such as those from eGeMAPS is to use a neural network to extract high level features from raw data and to use those automatically learned features in an acoustic feature vector for speech emotion recognition.

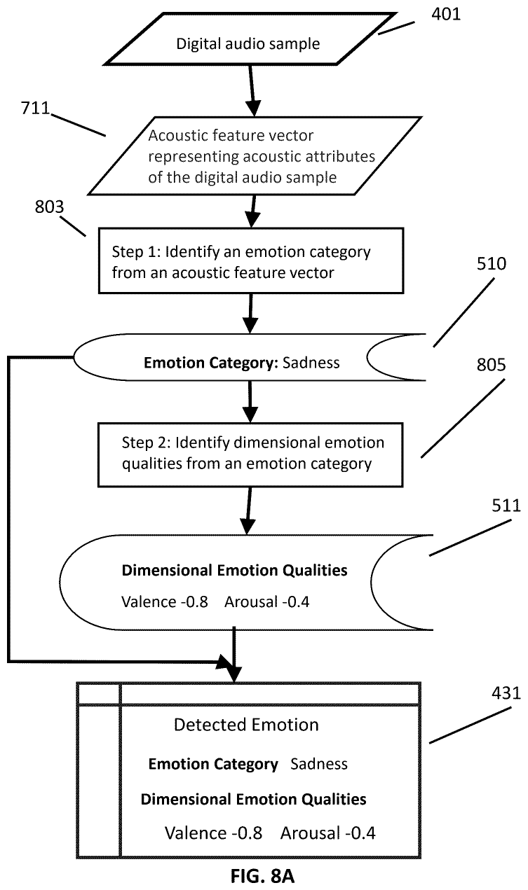

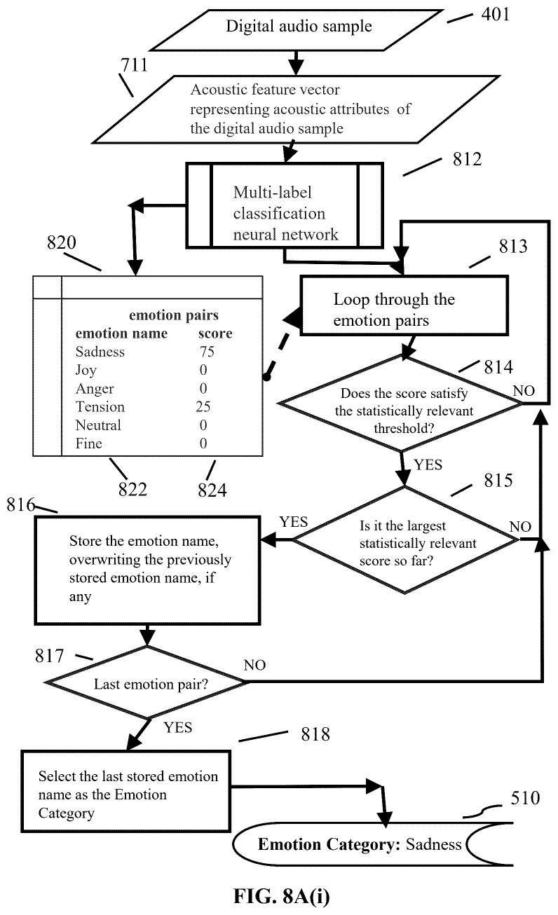

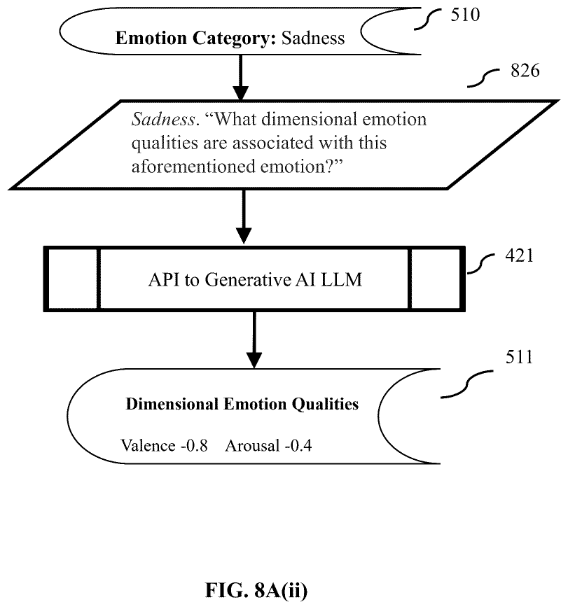

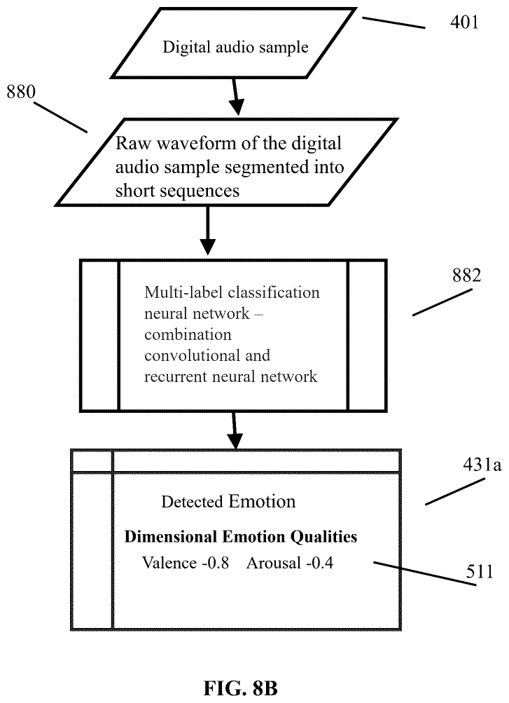

FIG. 8A depicts a flow diagram of a computer-implemented method of emotion detection model A in an exemplary embodiment. The method may comprise two steps. In the first step an acoustic feature vector 711 representing the digital audio sample 401 may be given as input to a process 803 that identifies an Emotion Category 610 from the acoustic feature vector 711. In the second step the name of the Emotion Category 610 is input to a process 805 that identifies Dimensional Emotion Qualities 511 from the name of the Emotion Category 610. The outputted ofDimensional Emotion Qualities 511 comprises the Detected Emotion 431.

FIG. 8A(i) depicts a flow diagram of a computer-implemented method for the first step in FIG. 8A, the process of identifying an Emotion Category 610 from an acoustic feature vector 711 representing the digital audio sample 401, in an exemplary embodiment. The acoustic features derived from human-defined features such as those from eGeMAPS may be used to form the acoustic feature vector 711. The acoustic feature vector 711 is given as input to a trained multi-label classification neural network 812. Said trained multi-label classification neural network 812 may be designed to use acoustic features such as those from eGeMAPS, as described in Mirsamadi and Barsoum's 2017 paper “Automatic speech emotion recognition using recurrent neural networks with local attention.”

In other embodiments, said trained multi-label classification neural network may be designed to use the raw waveform of the digital audio sample 401 segmented to sequences of up to 6 seconds to learn the acoustic features from which the acoustic feature vector is formed, although sequences of other lengths are possible. Trigeorgis et al.'s 2016 paper “Adieu features? End-to-end speech emotion recognition using a deep convolutional recurrent network” describes such a trained multi-label classification neural network.

The trained multi-label classification neural network 812 may produce a list 820 of emotion pairs, each pair comprising an emotion name 822 such as Sadness along with a score 824 for the emotion name, which score 824 is an estimate of the confidence with which the algorithm detects the named emotion 822 in the acoustic feature vector 711. The emotion names 822 and their scores 824 may be successively checked in a loop 813 to see, first, if the score 824 satisfies a predetermined statistically relevant threshold 814 and, if it does, if it is the largest score so far 815. The emotion name 822 associated with the largest such score 824 is stored within the loop 816. After all scores have been checked 817, the last stored emotion name may be selected 818 as the Emotion Category 610.

FIG. 8A(ii) depicts a flow diagram of a computer-implemented method for the second step in FIG. 8A, the process of identifying Dimensional Emotion Qualities 511 corresponding to an Emotion Category 610. The Emotion Category 610 is used as part of a prompt 826 given to an API to the Generative AI LLM 421. The prompt for the Emotion Category of Sadness is: “Sadness. What Dimensional Emotion Qualities are associated with this aforementioned emotion?” The Python code for an exemplary embodiment using ChatGPT 40 and the OpenAI Python API library is shown here:

```
chat_completion = client.chat.completions.create(
  messages=[
    {
      ″role″: ″user″,
      ″content″: Sadness. Convert the dimensional emotion qualities of the aforementioned
emotion to a numeric scale, using the framework proposed by Russell's Circumplex Model of
Affect and using a scale from −1 to 1 for valence and arousal. Report the results as ‘Valence’
followed by a number and ‘Arousal’ followed by a number.”,
    }
  ],
  model=″gpt-4-1106-preview″,
)
```

In an exemplary embodiment for the Emotion Category of Sadness, Dimensional Emotion Qualities 511 of a valance of −0.8 and arousal of −0.4 are output.

FIG. 8B depicts a flow diagram of a computer-implemented method for the emotion detection model B in an exemplary embodiment. The raw waveform 880 of the digital audio sample 401 may be segmented into sequences of up to 6 seconds, although sequences of other lengths are possible. In other embodiments, the acoustic feature vector 711 may be derived from human-defined features such as those from eGeMAPS. In the exemplary embodiment, the segmented raw waveform of the digital audio sample 401 may be given as input to a multi-label classification neural network 882, comprising a combination convolutional and recurrent neural network. Yang & Hirschberg's 2018 article, previously referenced, provides examples. A convolutional neural network identifies high-level acoustic features while a recurrent neural network captures temporal dependencies in the digital acoustic sample. The neural network outputs a Detected Emotion 431, comprising Dimensional Emotion Qualities 511.

Emotion detection models A and B are initially trained using selections of the follow datasets: CREMA-D, EmoSynth, JL Corpus, TESS, RECOLA, and SEMAINE, as described in the papers by Cao et al., 2104, Baird et al., 2018, Dupuis and Pichora-Fuller, 2010, and Mckeown et al., 2012, respectively. Emotion detection models A and B are periodically retrained with labeled digital audio input samples and updated.

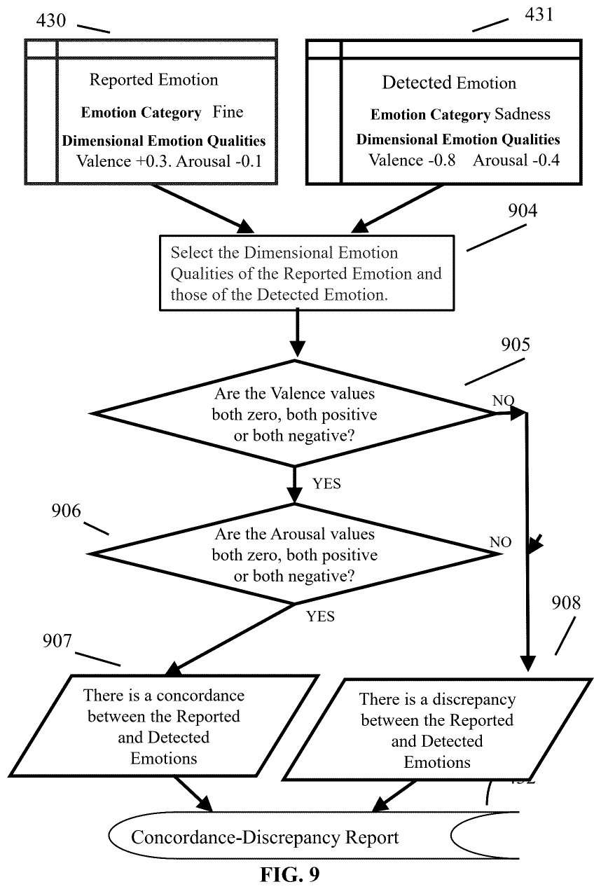

FIG. 9 depicts a flow diagram of a computer-implemented method for the concordance-discrepancy model in an exemplary embodiment. A Reported Emotion 430 and a Detected Emotion 431 are given as input to a process 904 that selects the Dimensional Emotion Qualities of both the Reported Emotion 430 and the Detected Emotion 431. A decision process 905 determines whether or not the valence values in the Reported Emotion 430 and the Detected Emotion 431 are in alignment, that is, both zero, both positive, or both negative. If the answer is Yes, a further process 906 determines whether or not the arousal values in the Reported Emotion 430 and the Detected Emotion 431 are in alignment, that is, both zero, both positive, or both negative. If the answer is Yes, there is a concordance between the Reported Emotion 430 and the Detected Emotion 431. If the answer to either decision process 905 or decision process 906 is No, there is a discrepancy between the Reported Emotion 430 and the Detected Emotion 431.

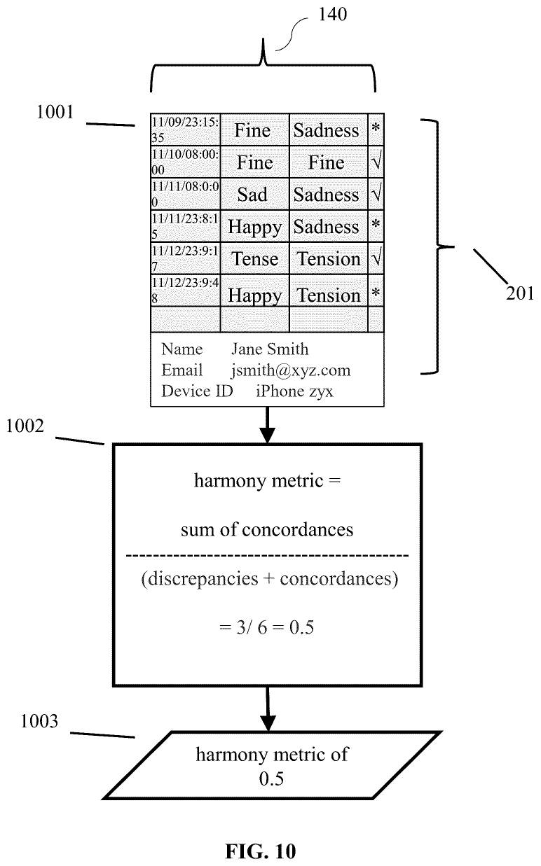

FIG. 10 depicts a flow diagram of a computer-implemented method for computing a harmony metric from the history of digital output reports 201 in an exemplary embodiment. The history of digital output reports 201 is given as input to a process that computes equation (1) from the plurality of Concordance-Discrepancy Reports 140 taken together.

$$harmony metric = ( sum of concordances ) / ( sum of concordances + sum of discrepancies ) \qquad ( 1 )$$

| | | | |
|---|---|---|---|
| 11/09/23:15:35 | Fine | Sadness | * |
| 11/10/08:00:00 | Fine | Fine | √ |
| 11/11/08:0:00 | Sad | Sadness | √ |
| 11/11/23:8:15 | Happy | Sadness | * |
| 11/12/23:9:17 | Tense | Tension | √ |
| 11/12/23:9:48 | Happy | Tension | * |

In the example for the exemplary embodiment, there are three asterisk marks (*) indicating discordances and three tick marks (√) indicating concordances. The harmony metric computed from the Concordance-Discrepancy Reports in the example is consequently computed as follows:

$$Harmony metric = 3 / ( 3 + 3 ) = 0.5 .$$

In other embodiments, a different equation may be used for the harmony metric to capture the extent to which the user's Reported Emotions and Detected Emotions are in alignment over time.

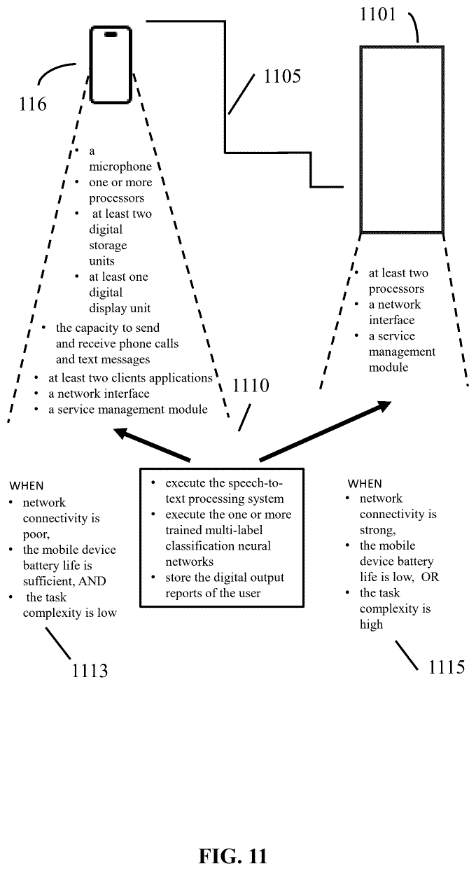

FIG. 11 depicts a system for generating a Reported Emotion, a Detected Emotion, and a Concordance-Discrepancy Report on the Reported Emotion and Detected Emotion, comprising a mobile device 116 associated with a user and a server 1101 with a connection 1105 to the mobile device.

The mobile device comprises a microphone, one or more processors, at least two digital storage units, at least one digital display unit, and the capacity to send and receive phone calls and text messages. The mobile device also comprises at least two client applications. One client application may accept spoken user input, display system outputs, send user data to the server, and send natural-language prompts to an API to a Generative AI LLM on the server. Another client may execute a speech-to-text processing system and one or more trained multi-label classification neural networks and further store digital output reports of a user. The mobile device also has a battery for providing power to the mobile device and a network interface for establishing a connection with the server that is configured to facilitate communication between the client application and the server. Additionally, the mobile device has a service management module configured to monitor the current network connectivity, monitor the battery life of the mobile device, and determine the complexity of the task to be processed. The service management module may also dynamically switch the execution of the speech-to-text processing system, the execution of the one or more trained multi-label classification neural networks, and the storage of digital output reports of the user, between the mobile device and the server based on the monitored network connectivity, mobile device battery life, and task complexity;

The server with a connection to the mobile device comprises a server processor configured to run the API to the Generative AI LLM, receive natural-language prompts for the AI LLM from the client application, and send responses back to the client application. The server processor is also configured to execute the speech-to-text processing system, execute the one or more trained multi-label classification neural networks, and store digital output reports of the user. The server processor further comprises a network interface for establishing the connection with the mobile device that is configured to facilitate communication between the client application and the mobile device. The server process further comprises a service management module configured to communicate with the mobile device to receive data regarding network connectivity, mobile device battery life, and task complexity. The service management module is also configured to accept from the mobile device the execution of the speech-to-text processing system and the one or more trained multi-label classification neural networks and to store the digital output reports of the user when determined to be optimal based on the received network connectivity data, mobile device battery life, and task complexity.

The system is configured to do the following 1110—execute the speech-to-text processing system, execute the one or more trained multi-label classification neural networks, and store the digital output reports of the user-on the mobile device 116 when the following 1113 holds: the network connectivity 1105 is poor, the mobile device battery life is sufficient, and the task complexity is low. The system is configured to do the following 1110—execute the speech-to-text processing system, execute the one or more trained multi-label classification neural networks, and store the digital output reports of the user—on the server 1101 when the following 1115 holds: the network connectivity 1105 is strong, the mobile device battery life is low, or the task complexity is high.

While the disclosure herein has been described in connection with specific embodiments, it is to be understood that the disclosure is not limited to the disclosed embodiments. On the contrary, the disclosure is intended to cover various modifications and equivalent arrangements included within the spirit and scope of the appended claims. Therefore, the description and drawings should be regarded as illustrative rather than restrictive. Additionally, any section headings used herein are for organizational purposes only and are not to be construed as limiting the subject matter described. The use of terms such as “including,” “comprising,” “having,” “containing,” or any other variation thereof, is intended to cover the items listed thereafter and equivalents thereof as well as additional items. All references cited herein are hereby incorporated by reference in their entirety.

What is claimed is:

1. A computer-implemented method to generate a Reported Emotion, a Detected Emotion, and a Concordance-Discrepancy Report, the method comprising:
i. based on an occurrence of a prompt, recording a digital audio sample representing an input utterance spoken by a user via a microphone of a mobile computing device associated with the user;
ii. generating the Reported Emotion by:
a. extracting via one or more processors from the digital audio sample a transcript comprising a sequence of natural-language words corresponding to the digital audio sample using speech-to-text processing; and
b. determining the Reported Emotion from the transcript using a natural-language processing model;
iii. generating the Detected Emotion by:
a. extracting a set of acoustic features via the one or more processors from the digital audio sample; and
b. processing the set of acoustic features to identify the Detected Emotion using an emotion detection model; and
iv. generating the Concordance-Discrepancy Report by:
a. analyzing the Reported Emotion and the Detected Emotion using a concordance-discrepancy model to determine if there is a concordance or a discrepancy between the Reported Emotion and the Detected Emotion; and
b. producing a Concordance-Discrepancy Report comprising the output of the concordance-discrepancy model.

2. The computer-implemented method of claim 1 whereby the mobile device comprises the microphone, the one or more processors, at least two digital storage units, and at least one digital display unit.

3. The computer-implemented method of claim 1 further comprising:
i. generating a digital output report comprising: (a) a unique identifier of the user's device, (b) the user's name, (c) the user's email address, (d) a timestamp indicating when the digital audio sample was received by the user's device, (e) the transcript, (f) the Reported Emotion, (g) the Detected Emotion, and (h) the Concordance-Discrepancy Report; and
ii. displaying for the user via the at least one display unit of the device associated with the user a Generative Artificial Intelligence (AI) Large Language Model (LLM) interpretation of the Concordance-Discrepancy Report.

4. The computer-implemented method of claim 3, wherein the Generative AI LLM interpretation of the Concordance-Discrepancy Report comprises the output from an application programming interface (API) to the Generative AI LLM using a natural-language prompt that asks for a clear and simple rewrite of the Concordance-Discrepancy Report.

5. The computer-implemented method of claim 3 further comprising repeating steps i and ii to produce a history of digital output reports and a history of Generative AI LLM interpretations of the Concordance-Discrepancy Reports.

6. The computer-implemented method of claim 1, wherein the prompt relates to one and only one of: (a) a phrase shown on the display unit of the device prompting the user to say what they are feeling, (b) the initiation of an outbound telephone call on the device, or (c) the acceptance of an inbound telephone call to the device.

7. The computer-implemented method of claim 1, wherein the set of acoustic features correspond to the extended Geneva Minimalistic Acoustic Parameter Set (eGeMAPS).

8. The computer-implemented method of claim 1, wherein the transcript of the input utterance spoken by the user contains a description of the emotion the user reports experiencing.

9. The computer-implemented method of claim 1, wherein the natural-language processing model comprises the following steps:
i. submitting the transcript to the API to the Generative AI LLM using a natural-language prompt asking which of a set of emotions most closely fits the transcript;
ii. selecting the output of the API to the Generative AI LLM as the Emotion Category;
iii. submitting the Emotion Category to the API to the Generative AI LLM using a prompt, the prompt asking which dimensional emotion qualities are associated with the aforementioned Emotion Category;
iv. selecting the output of the API to the Generative AI LLM as a plurality of Dimensional Emotion Qualities; and
v. storing the plurality of Dimensional Emotion Qualities as the Reported Emotion in the first of the at least two digital storage units.

10. The computer-implemented method of claim 1, wherein the emotion detection model comprises the following steps:
i. determining a feature vector corresponding to the digital audio sample, wherein the feature vector comprises the set of acoustic features:
ii. processing the feature vector as input to a trained multi-label classification neural network, the multi-label classification neural network configured to produce a plurality of emotion pairs, each emotion pair comprising an emotion name and an emotion score, wherein the emotion score of the emotion pair represents the probability that the digital audio sample expresses the named emotion of the emotion pair;
iii. processing the emotion pairs one by one in an outer loop in accordance with a predetermined statistically significant threshold by undertaking at least one of A, B or C for each iteration of the loop:
A. determining that the emotion score in the emotion pair satisfies the threshold, determining that the score is the optimal such score so far, and storing the emotion name in the second of the at least two digital storage units;
B. determining that the emotion score in the emotion pair satisfies the threshold, and determining that the score is not the optimal such score so far;
C. determining that the emotion score in the emotion pair does not satisfy the threshold;
iv. selecting the emotion name in the second of the at least two digital storage units as the Emotion Category;
v. submitting the Emotion Category to the API to the Generative AI LLM using a prompt, the prompt asking which dimensional emotion qualities are associated with the aforementioned Emotion Category:
vi. selecting the output of the API to the Generative AI LLM as the plurality of Dimensional Emotion Qualities; and
vii. storing the plurality of Dimensional Emotion Qualities as the Detected Emotion in the second of the at least two digital storage units.

11. The computer-implemented method of claim 1, wherein the emotion detection model comprises the following steps:
i. determining a feature vector corresponding to the digital audio sample, wherein the feature vector comprises the set of acoustic features;
ii. processing the feature vector as input to a trained multi-label classification neural network, the multi-label classification neural network configured to produce a plurality of Dimensional Emotion Qualities; and
iii. storing the plurality of Dimensional Emotion Qualities as the Detected Emotion in the second of the at least two digital storage units.

12. The computer-implemented method of claim 1, wherein the concordance-discrepancy model comprises the following steps:
i. selecting the plurality of Dimensional Emotion Qualities of the Reported Emotion;
ii. selecting the plurality of Dimensional Emotion Qualities of the Detected Emotion; and
iii. undertaking at least one of A or B:
A. determining that the Reported Emotion and Detected Emotion are in alignment with each other in terms of their Dimensional Emotion Qualities;
B. determining that the Reported Emotion and Detected Emotion are not in alignment with each other in terms of their Dimensional Emotion Qualities.

13. The computer implemented method of claim 5, wherein a harmony metric is computed by taking the ratio of discrepancies to the sum of discrepancies and concordances in the history of digital output reports.

14. A system for generating a Reported Emotion, a Detected Emotion, and a Concordance-Discrepancy Report on the Reported Emotion and the Detected Emotion, comprising a mobile device associated with a user and a server with a connection to the mobile device.

15. The system of claim 14, wherein the mobile device associated with the user comprises:
i. a microphone, one or more processors, at least two digital storage units, at least one digital display unit, and the capacity to send and receive phone calls and text messages;
ii. a client application on the mobile device configured to accept spoken user input, display system outputs, send user data to the server, and send natural-language prompts to an API to a Generative AI LLM on the server;
iii. a client application on the mobile device configured to execute a speech-to-text processing system and one or more trained multi-label classification neural networks, and to store digital output reports of the user;
iv. a battery for providing power to the mobile device;
v. a network interface for establishing a connection with the server and configured to facilitate communication between the client application and the server; and
vi. a service management module configured to:
a. monitor the current network connectivity;
b. monitor the battery life of the mobile device;
c. determine the complexity of the task to be processed; and
d. dynamically switch the execution of the speech-to-text processing system, the execution of the one or more trained multi-label classification neural networks, and the storage of digital output reports of the user between the mobile device and the server based on the monitored network connectivity, mobile device battery life, and task complexity.

16. The system of claim 14, wherein the server with a connection to the mobile device comprises:
i. a server processor configured to run an API to the Generative AI LLM, receive natural-language prompts for the AI LLM from the client application, and send responses back to the client application;
ii. a server processor configured to execute the speech-to-text processing system, execute the one or more trained multi-label classification neural networks, and store digital output reports of the user;
iii. a network interface for establishing the connection with the mobile device and configured to facilitate communication between the client application and the mobile device; and
iv. a service management module configured to:
a. communicate with the mobile device to receive data regarding network connectivity, mobile device battery life, and task complexity; and
b. accept the execution of the speech-to-text processing system and the one or more trained multi-label classification neural networks and the storage of the digital output reports of a user from the mobile device when determined to be optimal based on the received network connectivity data, mobile device battery life, and task complexity.

17. The system of claim 15 wherein the system is configured to execute the speech-to-text processing system, execute one or more trained multi-label classification neural networks, and store digital output reports of the user on the mobile device when the network connectivity is poor, the mobile device battery life is sufficient, and the task complexity is low.

18. The system of claim 16 wherein the system is configured to execute the speech-to-text processing system, execute one or more trained multi-label classification neural networks, and store digital output reports of the user on the server when the network connectivity is strong, the mobile device battery life is low, or the task complexity is high.
